(19) United States
(12) Patent Application Publication
(10) Pub. No.: US 2026/0330632 A1
(43) Pub. Date: Oct. 22, 2026

(54) CONTRAST-FREE AI-POWERED CORONARY ARTERY ASSESSMENT SYSTEM

(71) Applicant: Lucentia, Inc., New York, NY (US)

(72) Inventor: Sahar Jalal, New York, NY (US)

(21) Appl. No.: 19/653,601

(22) Filed: Apr. 21, 2026

Related U.S. Application Data
(60) Provisional application No. 63/791,922, filed on Apr. 21, 2025.

(51) Int. Cl. G06T 7/00; G06T 5/60; G06T 7/11; G16H 50/20

(52) U.S. Cl. CPC G06T 7/0012; G06T 5/60; G06T 7/11; G16H 50/20; G06T 2207/10081; G06T 2207/20084; G06T 2207/30101

(57) ABSTRACT

A system and method for generating enhanced coronary imaging representations from non-contrast computed tomography (CT) data using artificial intelligence. The system infers latent anatomical and structural information not directly observable in non-contrast CT input data — including vessel lumen clarity, soft plaque characteristics, and contrast-enhancement-related vessel features — enabling coronary assessment without contrast agent administration. The system comprises preprocessing modules, coronary artery localization and segmentation modules, image enhancement and synthesis modules, structural assessment modules, and output interface modules. The image enhancement and synthesis module applies trained transformation models to generate enhanced coronary representations using image-to-image transformation, latent space reconstruction, and feature-level enhancement. The system generates diagnostic outputs including plaque characterization, stenosis severity estimates, vessel geometry metrics, and uncertainty scores. The system integrates with existing PACS/RIS infrastructure via DICOM protocols and is deployable in cloud-based, on-premise, and edge configurations without new imaging hardware or contrast administration.

### CROSS-REFERENCE TO RELATED APPLICATIONS

This application claims priority to U.S. Provisional Application No. 63/791,922, filed Apr. 21, 2025, the entire contents of which are incorporated herein by reference.

### TECHNICAL FIELD

The present disclosure relates to coronary assessment system. In particular, the present disclosure relates to a contrast-free AI-powered coronary artery assessment system.

### BACKGROUND

Coronary artery disease (CAD) is the leading cause of death in the United States, accounting for approximately 600,000 deaths annually. Despite the significant clinical burden, early detection remains limited due to the constraints of existing diagnostic modalities.

Coronary Computed Tomography Angiography (CCTA) is currently the gold standard for non-invasive coronary imaging, providing detailed anatomical visualization of coronary vessels, plaque morphology, and stenosis severity. However, CCTA presents several barriers to widespread adoption:
   a. Contrast Requirement: CCTA requires intravenous administration of iodinated contrast agents, which carry risks including nephrotoxicity, allergic reactions, and contraindication in patients with renal insufficiency or prior allergic history.
   b. Cost and Access: CCTA costs approximately $1,500 per scan and requires specialized infrastructure, specialist staffing, and post-procedure monitoring, limiting its availability to a fraction of at-risk patients.
   c. Time Intensity: A complete CCTA examination typically requires 45 minutes or more including patient preparation, contrast injection, scan acquisition, and post-procedure monitoring.
   d. Patient Exclusion: A significant proportion of at-risk patients are excluded from CCTA due to contraindications related to renal function, contrast allergies, heart rate, or body habitus.

Coronary Artery Calcium (CAC) scoring scans are widely performed, low-cost, non-contrast CT scans used for cardiovascular risk stratification. However, CAC scans are limited in diagnostic value because they cannot detect non-calcified soft plaque — the type of plaque most associated with acute coronary events and rupture risk. Prior systems, including those described in US20230132940A1 (Cleerly), have attempted AI-based analysis of non-contrast CT data, but are limited to risk stratification, plaque classification, and calcium scoring from existing image features. These prior systems do not infer or reconstruct latent anatomical or structural information not directly observable in non-contrast CT data, and do not generate full contrast-equivalent or diagnostically enriched coronary artery imagery enabling direct radiological interpretation comparable to CCTA.

A significant unmet clinical need therefore exists for a system and method that: (i) accepts non-contrast CT scans as input; (ii) generates diagnostically enriched coronary imaging representations that provide comparable diagnostic information to contrast-enhanced CCTA; (iii) provides plaque characterization and stenosis assessment; and (iv) integrates into existing clinical workflows without requiring new imaging equipment, contrast administration, or specialist staffing. The present disclosure addresses all of these unmet needs.

### SUMMARY

The present disclosure provides a contrast-free AI-powered coronary artery assessment system and method that generates enhanced, augmented, or diagnostically enriched coronary imaging representations from non-contrast CT data without the administration of contrast agents. A key aspect of the disclosure is the inference of latent anatomical and structural information — including vessel lumen clarity, plaque morphology, and perfusion-related vessel characteristics — that is not directly observable in non-contrast CT input data.

In one aspect, the disclosure provides a system configured to derive enhanced coronary representations from non-contrast CT data, wherein the enhanced representations include anatomical or structural information not directly observable in the input imaging data.

In another aspect, the disclosure provides a system for generating contrast-equivalent, enhanced, augmented, or diagnostically enriched coronary imaging representations from non-contrast CT data, comprising: (a) one or more preprocessing modules; (b) one or more coronary artery localization and segmentation modules; (c) one or more image enhancement and synthesis modules; (d) one or more structural assessment modules; and (e) one or more output interface modules integrated with clinical imaging infrastructure.

In another aspect, the disclosure provides a method for generating enhanced coronary representations from non-contrast CT data comprising: receiving non-contrast CT imaging data; preprocessing the imaging data; localizing and segmenting coronary arterial structures; generating enhanced coronary imagery including information inferred from but not directly observable in the input data; performing structural assessment; and delivering diagnostic results to clinical imaging systems.

In various embodiments, the system and method enable: (i) plaque characterization including calcified, non-calcified, and mixed plaque types; (ii) stenosis severity estimation on a per-vessel-segment basis; (iii) interactive three-dimensional coronary tree visualizations; (iv) uncertainty quantification; (v) longitudinal comparison; and (vi) integration into outpatient, inpatient, and emergency settings without new hardware. The system may be extended to generate additional diagnostic outputs, biomarkers, or predictive indicators derived from non-contrast imaging data, including outputs not yet identified at the time of filing.

The system operates within clinically relevant timeframes without contrast agents, intravenous access, specialist staffing, post-procedure monitoring, or modification to existing CT hardware.

The above and other preferred features, including various details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures(or drawings). A brief introduction of the figures is below.

FIG. 1 illustrates a block diagram of a contrast free AI-powered coronary artery assessment system;

FIG. 2 illustrates an embodiment of a training data pipeline;

FIG. 3 illustrates an example of a clinical workflow;

FIG. 4 illustrates various photographs of anatomical scans;

FIG. 5 illustrates an embodiment of a structural assessment output;

FIG. 6 illustrates a flowchart of an embodiment of a method of generating enhanced coronary imaging representations from non-contrast CT data; and

FIG. 7 illustrates a computing system that may be used in systems and methods described herein.

### DETAILED DESCRIPTION

Reference will now be made in detail to several embodiments, examples of which are illustrated in the accompanying figures. The Figures(Figs.) and the following description relate to preferred embodiments by way of illustration only. It is noted that wherever practicable similar or like reference numbers may be use in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

The present disclosure provides several technical advantages over existing coronary artery assessment systems and methods. First, the system and method of the present disclosure enable generation of diagnostically enriched coronary imaging representations from non-contrast CT data, thereby eliminating the requirement for intravenous administration of iodinated contrast agents and the associated risks of nephrotoxicity, allergic reactions, and contraindication in patients with renal insufficiency or prior allergic history. Second, the system and method of the present disclosure infer latent anatomical and structural information, including vessel lumen clarity, soft tissue contrast characteristics, plaque morphology, and perfusion-related vessel characteristics, that is not directly observable in or recoverable through conventional processing of non-contrast CT input data, thereby providing clinical information comparable to contrast-enhanced coronary CT angiography (CCTA) without requiring contrast administration. Third, the system and method of the present disclosure operate on non-contrast CT imaging data already routinely acquired in clinical practice, including coronary artery calcium (CAC) scoring scans and low-dose chest CT scans, thereby enabling coronary artery disease assessment without requiring additional imaging acquisitions, new CT scanner hardware, or modification to existing imaging infrastructure. Fourth, the system and method of the present disclosure operate within clinically relevant timeframes without requiring specialist staffing, intravenous access, or post-procedure monitoring, thereby reducing per-patient cost, expanding patient throughput, and enabling deployment in clinical settings where CCTA is impractical or unavailable. Fifth, the system and method of the present disclosure expand the eligible patient population for coronary artery disease assessment by enabling diagnostic-quality coronary evaluation for patients who are contraindicated for contrast-enhanced imaging, including patients with renal insufficiency, contrast allergies, or other clinical contraindications that would otherwise preclude CCTA. Sixth, the system and method of the present disclosure enable repeated longitudinal coronary assessment without cumulative contrast exposure, thereby supporting monitoring of disease progression and treatment response over time. Seventh, the system and method of the present disclosure are deployable across multiple configurations, including cloud-based, on-premise, and edge deployment, and are compatible with existing clinical imaging infrastructure, including picture archiving and communication systems (PACS), radiology information systems (RIS), and electronic health record systems, via standard DICOM-compatible interfaces.

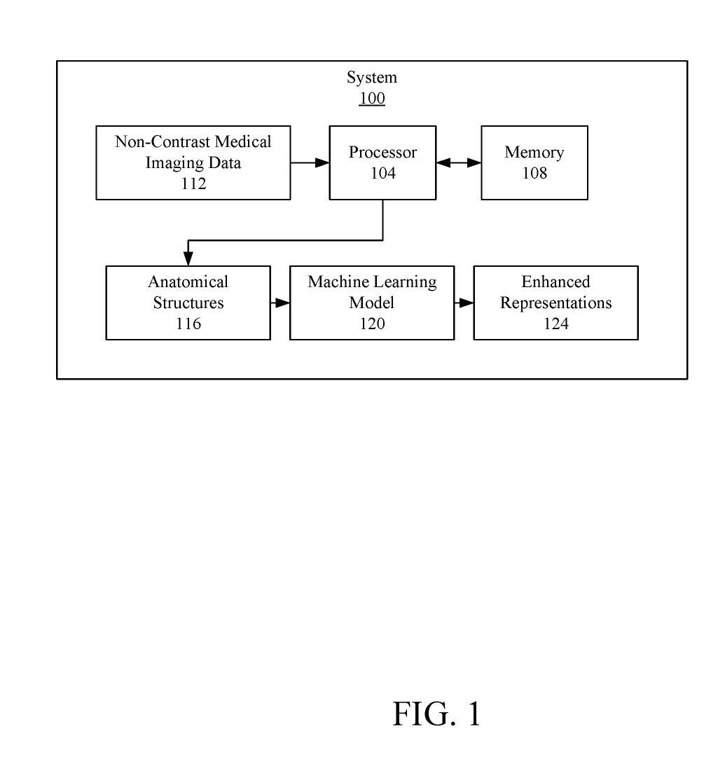

Referring now to FIG. 1, a block diagram of a contrast free AI-powered coronary artery assessment system is presented. System 100 may include processor 104 and memory 108. Processor 104 may include one or more central processing units (CPUs), graphics processing units (GPUs), tensor processing units (TPUs), application-specific integrated circuits (ASICs), field-programmable gate arrays (FPGAs), digital signal processors (DSPs), or any combination thereof. Memory 108 may include one or more of random access memory (RAM), read-only memory (ROM), flash memory, solid-state drives (SSDs), hard disk drives (HDDs), or any other non-transitory computer-readable storage medium. Processor 104 and memory 108 may be communicatively coupled via one or more system buses, point-to-point interconnects, shared memory interfaces, or any other suitable communication pathway.

In some embodiments, processor 104 may be configured to receive non-contrast medical imaging data 112. Non-contrast medical imaging data 112 may include one or more of cardiac CT scan data, coronary artery calcium (CAC) scoring scan data, low-dose chest CT data, non-cardiac chest CT data in which coronary structures appear incidentally, multi-energy CT data, or any other CT imaging data acquired without administration of a contrast agent to a patient. Non-contrast medical imaging data 112 may be formatted in accordance with the Digital Imaging and Communications in Medicine (DICOM) standard or any other suitable medical imaging data format. Processor 104 may receive non-contrast medical imaging data 112 via one or more communication interfaces, including but not limited to a network interface communicatively coupled to a picture archiving and communication system (PACS), a radiology information system (RIS), a direct connection to a CT imaging system, a local or remote file system, a cloud-based storage service, or any combination thereof. In some embodiments, processor 104 may receive non-contrast medical imaging data 112 in real-time or near-real-time from a CT imaging system, or may retrieve previously acquired non-contrast medical imaging data 112 from a storage system.

Processor 104 may be configured to identify anatomical structures 116 from non-contrast medical imaging data 112. In some embodiments, processor 104 may identify anatomical structures 116 by applying one or more machine learning models, including but not limited to convolutional neural networks (CNNs), U-Net architectures, transformer-based segmentation models, region-based convolutional neural networks, or any combination thereof, trained to detect and delineate anatomical features within non-contrast CT imaging data. Anatomical structures 116 may include one or more of coronary arterial structures, coronary artery segments, vessel walls, vessel lumens, calcified deposits, pericardial boundaries, cardiac chambers, the aortic root, or any other anatomically relevant structures identifiable within non-contrast medical imaging data 112. In some embodiments, processor 104 may identify anatomical structures 116 by performing one or more of voxel-level segmentation, region-of-interest extraction, centerline extraction, three-dimensional volumetric reconstruction, multi-planar reformatting, or any combination thereof.

Processor 104 may apply one or more preprocessing operations to non-contrast medical imaging data 112 prior to identifying anatomical structures 116, including but not limited to spatial normalization, intensity normalization, noise reduction, motion artifact correction, field-of-view cropping, or resampling to a target voxel resolution. In some embodiments, processor 104 may identify anatomical structures 116 using a multi-stage pipeline in which a first model localizes a region of interest within non-contrast medical imaging data 112 and a second model performs fine-grained segmentation of anatomical structures 116 within the localized region. For instance, in one embodiment, a first machine learning model may receive non-contrast medical imaging data 112 including a full thoracic CT volume and generate a bounding volume or probability map identifying the cardiac region of interest, including the pericardial boundary and aortic root, thereby reducing the spatial extent of the imaging data to a cardiac-specific sub-volume; a second machine learning model may then receive the cardiac-specific sub-volume and perform fine-grained segmentation of coronary arterial structures therein, including detection of coronary ostia, tracing of coronary centerlines, delineation of vessel lumen and vessel wall boundaries, and identification of calcified deposits along the coronary tree, thereby producing voxel-level segmentation maps of anatomical structures 116 at higher spatial precision than would be achievable by applying a single model to the full thoracic CT volume. In some embodiments, processor 104 may be configured to preprocess non-contrast medical imaging data 112 to produce standardized input suitable for downstream processing. Non-contrast medical imaging data 112 may be received in DICOM format or any other suitable medical imaging data format. Preprocessing operations may include, but are not limited to: Hounsfield unit windowing and normalization; voxel resampling to a standardized isotropic resolution; field-of-view cropping; motion artifact reduction; noise reduction; and image quality assessment. In some embodiments, processor 104 may be configured to preprocess non-contrast medical imaging data 112 received from a variety of CT scanner models and manufacturers without requiring scanner-specific calibration. In various embodiments, processor 104 may additionally be configured to accept and preprocess: low-dose cardiac CT data; non-cardiac chest CT data in which coronary structures appear incidentally; multi-energy CT data; and other CT-based imaging modalities. Processor 104 may be configured to identify and isolate the cardiac region within the imaging field of view regardless of whether the scan was acquired specifically for coronary assessment. For instance, in one embodiment, processor 104 may receive non-cardiac chest CT data acquired for pulmonary embolism evaluation or lung cancer screening, and may automatically detect the cardiac silhouette and coronary arterial structures within the thoracic field of view, extract a cardiac-specific sub-volume encompassing the coronary vasculature, and process the extracted sub-volume through the downstream localization, segmentation, and enhancement pipeline to generate enhanced representations 124, thereby enabling opportunistic coronary artery disease assessment from imaging data originally acquired for a non-cardiac clinical indication.

In some embodiments, system 100 may include a localization and segmentation module stored in memory 108 and executable by processor 104. The localization and segmentation module may be configured to receive preprocessed non-contrast medical imaging data 112 and identify the anatomical location of coronary arterial structures within non-contrast medical imaging data 112. Processor 104 may be configured to execute one or more machine learning models within the localization and segmentation module to identify anatomical structures 116 from non-contrast medical imaging data 112. One or more machine learning models within the localization and segmentation module may be configured to detect coronary ostia, trace coronary centerlines, segment the coronary lumen and vessel wall, and identify calcified deposits within the coronary tree. Machine learning models employed by processor 104 within the localization and segmentation module to identify anatomical structures 116 may include, without limitation: convolutional neural networks; attention-based architectures; graph neural networks; hybrid architectures; and other architectures configured to perform three-dimensional semantic segmentation of medical imaging data. In some embodiments, the one or more machine learning models within the localization and segmentation module may be trained using one or more training datasets comprising annotated non-contrast CT imaging data in which coronary arterial structures have been delineated by expert annotators, automated annotation pipelines, or a combination thereof. Training datasets may include voxel-level ground truth annotations for one or more of coronary ostia locations, coronary centerline trajectories, vessel lumen boundaries, vessel wall boundaries, calcified deposit regions, and coronary segment labels. In some embodiments, the one or more machine learning models within the localization and segmentation module may be trained using one or more of supervised learning, semi-supervised learning, self-supervised learning, transfer learning, or any combination thereof. In some embodiments, the one or more machine learning models within the localization and segmentation module may be trained using one or more loss functions configured to optimize segmentation accuracy, including without limitation cross-entropy loss, Dice loss, focal loss, boundary loss, or any combination thereof. In some embodiments, training datasets used to train the one or more machine learning models within the localization and segmentation module may include imaging data acquired across multiple clinical institutions, CT scanner platforms, and patient populations to promote generalization and robustness of the trained models across scanner-to-scanner variability and diverse patient anatomies.

Referring still to FIG. 1, processor 104 may be configured to provide anatomical structures 116 to machine learning model 120. In some embodiments, anatomical structures 116 may be provided to machine learning model 120 as one or more of segmentation masks, volumetric regions of interest, extracted centerline representations, three-dimensional mesh representations, voxel-level label maps, cropped sub-volumes corresponding to identified coronary arterial structures, or any combination thereof. Machine learning model 120 may be stored in memory 108 and executed by processor 104. In some embodiments, processor 104 may provide anatomical structures 116 to machine learning model 120 together with corresponding portions of non-contrast medical imaging data 112, such that machine learning model 120 receives both the identified anatomical structures 116 and the underlying imaging data from which they were derived. In some embodiments, processor 104 may provide anatomical structures 116 to machine learning model 120 as input tensors, feature vectors, multi-channel volumetric arrays, or any other data representation suitable for processing by machine learning model 120.

Machine learning model 120 may be configured to process anatomical structures 116 to generate enhanced representations 124. Enhanced representations 124 may include one or more enriched coronary imaging representations that include anatomical or structural information not directly observable in or recoverable through conventional processing of non-contrast medical imaging data 112. In some embodiments, enhanced representations 124 may identify, infer, or reconstruct one or more of: vessel lumen clarity, including delineation of lumen boundaries and intraluminal features; soft tissue contrast characteristics, including differentiation of tissue densities and attenuation profiles approximating those obtainable through contrast-enhanced imaging; plaque-related features, including identification and characterization of calcified plaque, non-calcified plaque, mixed plaque, fibroatheroma, fibrocalcific plaque, pathological intimal thickening, or adaptive intimal thickening; stenosis severity on a per-vessel-segment basis; perfusion-related vessel characteristics; vessel wall morphology; or any other anatomical, structural, or diagnostic features that are absent from, latent within, or not directly discernible in non-contrast CT acquisitions. In some embodiments, machine learning model 120 may generate enhanced representations 124 by inferring latent anatomical information from patterns, spatial relationships, and structural cues present within anatomical structures 116 and non-contrast medical imaging data 112, thereby producing diagnostically enriched imagery that provides clinical information comparable to contrast-enhanced coronary CT angiography (CCTA) without requiring administration of a contrast agent to a patient.

In some embodiments, machine learning model 120 may employ one or more transformation approaches to generate enhanced representations 124, including without limitation: (i) direct image-to-image transformation, wherein machine learning model 120 maps non-contrast input volumes to contrast-enhanced output volumes at a pixel-level or voxel-level; (ii) latent space reconstruction, wherein machine learning model 120 encodes non-contrast input data into a learned latent representation and decodes the latent representation into enhanced output imagery; (iii) feature-level enhancement, wherein intermediate feature representations extracted from non-contrast input data are selectively augmented or refined to produce enhanced output; and (iv) hybrid multi-stage transformation, wherein two or more of the foregoing approaches are combined in a sequential or parallel pipeline. In some embodiments, machine learning model 120 may be trained using one or more training datasets to learn mappings between non-contrast CT imaging data and corresponding contrast-enhanced or ground truth anatomical representations. Training datasets may include one or more of: (i) ex-vivo paired datasets, wherein cadaveric cardiac specimens are imaged under both non-contrast and contrast-enhanced conditions, with co-registered ground truth obtained from complementary analytical modalities including micro-CT imaging, histopathological analysis, or any combination thereof, thereby providing sub-millimeter precision ground truth for coronary vessel structures and plaque composition; (ii) in-vivo paired datasets, comprising coronary artery calcium (CAC) scoring CT scans and coronary CT angiography (CCTA) scans acquired from the same patients within a clinically relevant time interval, enabling learning of mappings between non-contrast and contrast-enhanced coronary appearances across diverse patient anatomies and disease states; and (iii) multi-site and multi-scanner datasets, comprising imaging data acquired across multiple clinical institutions and CT scanner platforms, enabling generalization across scanner-to-scanner variability encountered in real-world deployment. In some embodiments, machine learning model 120 may be trained using one or more of supervised learning, semi-supervised learning, self-supervised learning, unsupervised learning, adversarial training, or any combination thereof. In some embodiments, when training datasets include unpaired imaging data in which non-contrast input samples and contrast-enhanced or ground truth target samples are not acquired from the same patient or specimen and lack pixel-level or voxel-level spatial correspondence, machine learning model 120 may be trained using one or more unpaired learning paradigms, including without limitation: cycle-consistent adversarial training, wherein a pair of generator networks learns forward and inverse mappings between non-contrast and contrast-enhanced imaging domains subject to a cycle-consistency constraint that enforces reconstruction fidelity; contrastive learning, wherein the model learns domain-invariant feature representations by maximizing similarity between corresponding anatomical features across imaging domains while minimizing similarity between non-corresponding features; domain adaptation, wherein the model learns to transfer knowledge from a source imaging domain to a target imaging domain by aligning feature distributions across domains through adversarial alignment, distribution matching, or feature-space regularization; or any combination thereof. In some embodiments, machine learning model 120 may be trained using one or more loss functions, including without limitation perceptual loss, adversarial loss, pixel-wise reconstruction loss, structural similarity loss, feature matching loss, cycle-consistency loss, contrastive loss, or any combination thereof, configured to optimize the fidelity, diagnostic quality, and anatomical accuracy of enhanced representations 124 relative to corresponding ground truth data.

In some embodiments, system 100 may include a structural assessment module stored in memory 108 and executable by processor 104. The structural assessment module may be configured to receive enhanced representations 124 and perform quantitative analysis of coronary artery structures identified therein. The structural assessment module may generate one or more of the following diagnostic outputs: (i) plaque characterization, including classification of detected plaque as calcified, non-calcified, mixed, or other subtypes, including fibroatheroma, fibrocalcific plaque, pathological intimal thickening, and adaptive intimal thickening; (ii) stenosis estimation, including quantitative estimation of luminal narrowing on a per-vessel-segment basis, including percent diameter stenosis and area stenosis with severity classification; (iii) vessel geometry metrics, including vessel diameter, wall thickness, lumen area, and centerline geometry along the coronary tree; (iv) coronary tree visualization, including interactive two-dimensional and three-dimensional visualizations with overlaid structural findings; (v) uncertainty quantification, including confidence scores or uncertainty estimates associated with enhanced representations 124 and structural assessment outputs; and (vi) longitudinal comparison, including co-registration and comparative analysis across multiple time-point studies to identify changes in plaque burden, stenosis severity, and vessel geometry. In some embodiments, the structural assessment module may generate additional diagnostic outputs, biomarkers, or predictive indicators as the system is extended, without departing from the scope of the present disclosure.

In some embodiments, system 100 may include an output interface module stored in memory 108 and executable by processor 104. The output interface module may be configured to deliver enhanced representations 124 and structural assessment results generated by system 100 to one or more clinical information systems. The output interface module may be compatible with standard Digital Imaging and Communications in Medicine (DICOM) protocols and may be configured to integrate with one or more of a hospital picture archiving and communication system (PACS), a radiology information system (RIS), an electronic health record system, a radiology reporting platform, or any combination thereof. In some embodiments, the output interface module may provide a programmatic application programming interface (API) configured to receive imaging data and return processed outputs, thereby enabling integration with third-party platforms, including downstream AI-based analysis tools. Output formats supported by the output interface module may include, without limitation: DICOM-formatted imaging series; structured reports compatible with Health Level Seven Fast Healthcare Interoperability Resources (HL7 FHIR) standards; web-based interactive viewer outputs; and PDF-formatted diagnostic summary reports. In some embodiments, the output interface module may support additional output formats or integration protocols as the system is extended, without departing from the scope of the present disclosure.

In some embodiments, system 100 may be deployable in one or more deployment configurations, including without limitation: (i) a cloud-based configuration, wherein non-contrast medical imaging data 112 is securely transmitted to a cloud-based processing platform for processing by system 100; (ii) an on-premise configuration, wherein system 100 is installed on local hospital or imaging center hardware within the institution's existing information technology infrastructure; (iii) an edge computing configuration, wherein system 100 is executed on hardware co-located with or integrated into a CT scanner, enabling real-time or near-real-time processing at the point of image acquisition; or (iv) any combination thereof. In some embodiments, system 100 may generate enhanced representations 124 and associated diagnostic outputs within clinically relevant timeframes, without requiring new CT scanner hardware, contrast agent administration, specialist staffing, intravenous access, or post-procedure monitoring.

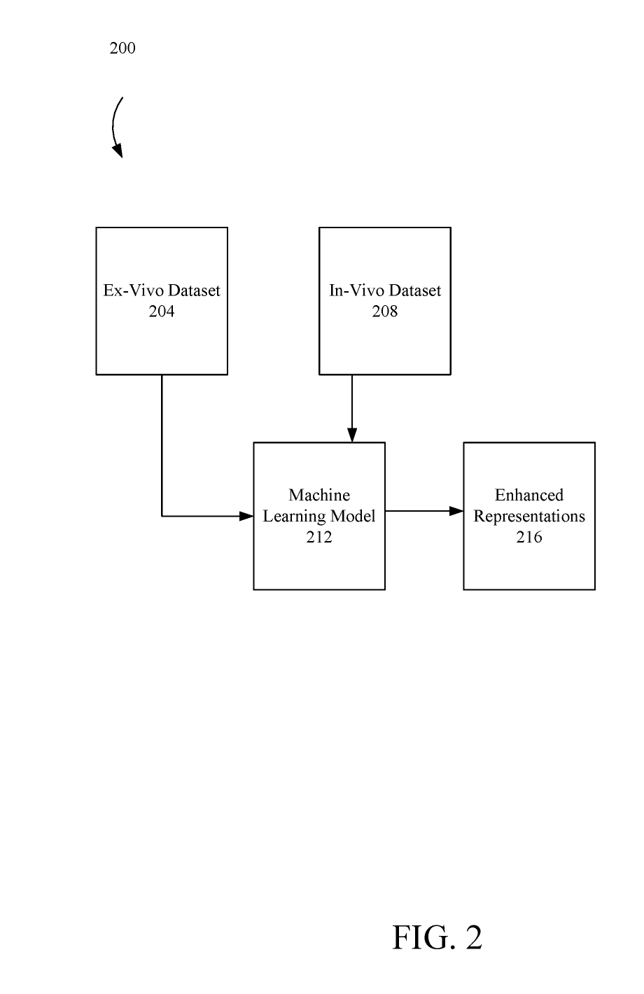

Referring now to FIG. 2, a training data pipeline for a machine learning model is presented. In some embodiments, machine learning model 212 may be trained using one or more training datasets, including ex-vivo dataset 204 and in-vivo dataset 208. Ex-vivo dataset 204 may include imaging data acquired from cadaveric specimens, including non-contrast CT imaging data and corresponding co-registered ground truth data obtained through contrast-enhanced imaging, micro-CT imaging, histopathological analysis, or any combination thereof. Ex-vivo dataset 204 may provide high-fidelity anatomical ground truth for coronary vessel morphology, plaque composition, and lumen geometry under controlled imaging conditions. In-vivo dataset 208 may include clinical imaging data acquired from living patients, including paired or unpaired non-contrast CT imaging data and contrast-enhanced coronary CT angiography (CCTA) data. In some embodiments, in-vivo dataset 208 may include coronary artery calcium (CAC) scoring scan data paired with corresponding CCTA data acquired from the same patient within a clinically relevant time interval. Machine learning model 212 may be trained on ex-vivo dataset 204, in-vivo dataset 208, or any combination thereof, to learn mappings between non-contrast CT imaging data and corresponding contrast-enhanced or ground truth anatomical representations. In some embodiments, machine learning model 212 may be trained using one or more of supervised learning, semi-supervised learning, self-supervised learning, unsupervised learning, adversarial training, or any combination thereof. Upon completion of training, machine learning model 212 may be configured to generate enhanced representations 216 from non-contrast CT imaging data. Enhanced representations 216 may include diagnostically enriched coronary imaging representations that include anatomical or structural information inferred from but not directly observable in the non-contrast input data. In some embodiments, machine learning model 212 may correspond to machine learning model 120 of FIG. 1, and enhanced representations 216 may correspond to enhanced representations 124 of FIG. 1.

In some embodiments, machine learning model 212 may be trained on datasets including paired and/or unpaired imaging data from multiple sources and modalities. Training datasets may include, without limitation: (i) ex-vivo paired datasets, wherein ex-vivo cadaver cardiac specimens are imaged under both non-contrast and contrast-enhanced conditions, with co-registered ground truth obtained from complementary analytical modalities, thereby providing sub-millimeter precision ground truth for coronary vessel structures and plaque composition; (ii) in-vivo paired datasets, comprising coronary artery calcium (CAC) scoring CT scans and coronary CT angiography (CCTA) scans acquired from the same patients, enabling learning of mappings between non-contrast and contrast-enhanced coronary appearances across diverse patient anatomies and disease states; and (iii) multi-site and multi-scanner datasets, including imaging data acquired across multiple clinical institutions and CT scanner platforms, enabling generalization across scanner-to-scanner variability encountered in real-world deployment. In some embodiments, ex-vivo paired datasets may correspond to ex-vivo dataset 204 and in-vivo paired datasets may correspond to in-vivo dataset 208. The use of training data from heterogeneous sources and acquisition protocols may improve the robustness and generalizability of machine learning model 212 across clinical environments without requiring scanner-specific calibration.

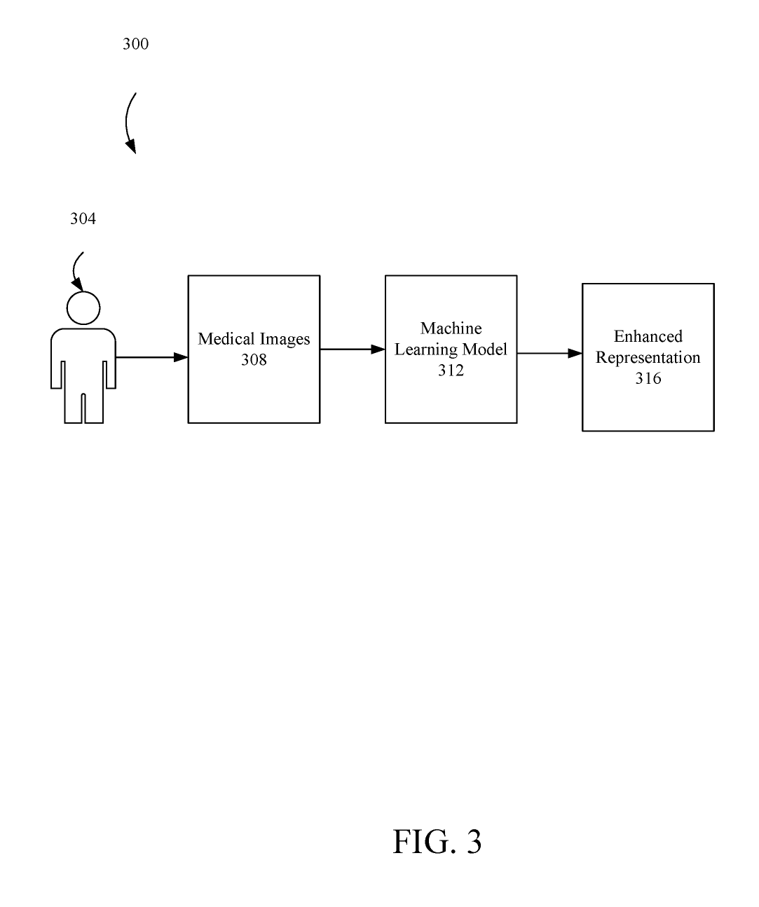

Referring now to FIG. 3, an example of a clinical workflow for generating enhanced coronary imaging representations is presented. In some embodiments, medical images 308 of patient 304 may be received by machine learning model 312. Medical images 308 may include non-contrast CT imaging data acquired during a clinical encounter, including but not limited to coronary artery calcium (CAC) scoring scan data, low-dose cardiac CT data, non-cardiac chest CT data in which coronary structures appear incidentally, or any other non-contrast CT imaging data. In some embodiments, medical images 308 may be received from a CT imaging system, a picture archiving and communication system (PACS), a radiology information system (RIS), or any other clinical imaging data source. Machine learning model 312 may be configured to process medical images 308 to generate enhanced representation 316. Enhanced representation 316 may include one or more diagnostically enriched coronary imaging representations that include anatomical or structural information inferred from but not directly observable in medical images 308. In some embodiments, enhanced representation 316 may provide clinical information comparable to contrast-enhanced coronary CT angiography (CCTA) without requiring administration of a contrast agent to a patient. In some embodiments, machine learning model 312 may correspond to machine learning model 120 of FIG. 1 or machine learning model 212 of FIG. 2, and enhanced representation 316 may correspond to enhanced representations 124 of FIG. 1 or enhanced representations 216 of FIG. 2. The clinical workflow illustrated in FIG. 3 may be performed within clinically relevant timeframes, thereby enabling integration into existing clinical workflows without requiring additional specialist staffing, contrast administration, or modification to existing CT hardware.

In some embodiments, the clinical workflow illustrated in FIG. 3 may be implemented in one or more deployment configurations, including without limitation: (i) cloud-based deployment, wherein non-contrast CT imaging data, including DICOM-formatted data, is securely transmitted to a cloud-based processing platform hosting machine learning model 312, and wherein enhanced representation 316 and any associated diagnostic outputs are returned to the originating clinical system via secure network communication; (ii) on-premise deployment, wherein machine learning model 312 is installed on local hospital or imaging center hardware within the institution's existing information technology infrastructure, enabling processing of medical images 308 without transmitting imaging data outside the institutional network; (iii) edge deployment, wherein machine learning model 312 is executed on hardware co-located with or integrated into the CT scanner, enabling real-time or near-real-time processing of medical images 308 at the point of image acquisition; and (iv) application programming interface (API) integration, wherein a programmatic interface is provided to enable integration with third-party clinical software, radiology information systems, hospital workflow management systems, or any combination thereof. In some embodiments, the clinical workflow illustrated in FIG. 3 may be implemented using a combination of the foregoing deployment configurations without departing from the scope of the present disclosure.

In some embodiments, the clinical workflow illustrated in FIG. 3 may be applied in one or more clinical use case settings, including without limitation: (i) population-level cardiac screening, wherein large-scale screening programs are applied to asymptomatic patients at cardiovascular risk using non-contrast CT scans already acquired for calcium scoring or other purposes; (ii) pre-imaging triage, wherein medical images 308 are processed to identify patients requiring urgent intervention and to distinguish such patients from those manageable without additional invasive or contrast-enhanced procedures; (iii) contrast-contraindicated patient assessment, wherein diagnostic-quality coronary assessment is provided for patients contraindicated for contrast-enhanced imaging due to renal insufficiency, contrast allergy, or other clinical contraindications; (iv) emergency cardiac assessment, wherein rapid coronary assessment is generated from existing or expedited non-contrast CT acquisitions in clinical scenarios where time constraints preclude standard coronary CT angiography (CCTA); (v) incidental finding assessment, wherein non-cardiac CT scans in which coronary structures appear incidentally are processed to generate enhanced representation 316 and associated diagnostic outputs; and (vi) longitudinal disease monitoring, wherein repeated non-invasive coronary assessment is performed without contrast exposure, thereby supporting monitoring of disease progression, treatment response, and temporal changes in plaque burden or stenosis severity. In some embodiments, the clinical workflow illustrated in FIG. 3 may be applied in additional clinical use case settings beyond those expressly identified herein without departing from the scope of the present disclosure.

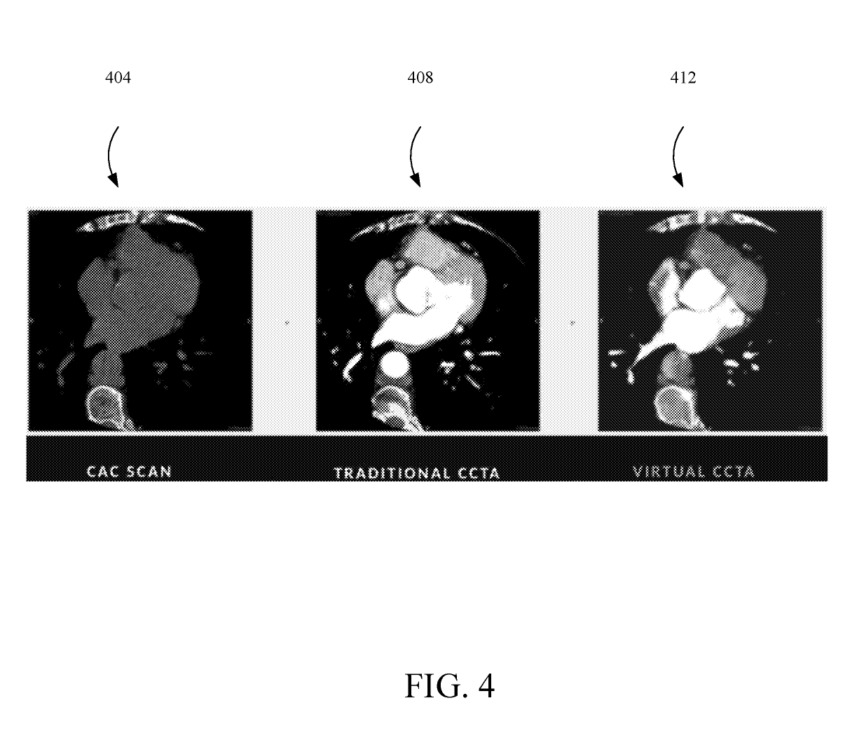

Referring now to FIG. 4, photographs of various anatomical scans are presented. FIG. 4 illustrates a representative input/output comparison comprising three coronary imaging photographs. A first photograph (left) depicts a non-contrast coronary artery calcium (CAC) scan 404 serving as input to the system, in which coronary arterial structures are visible with limited soft tissue contrast and without lumen delineation characteristic of non-contrast CT acquisitions. A second photograph (center) depicts a traditional contrast-enhanced coronary CT angiography (CCTA) scan 408 acquired with intravenous administration of an iodinated contrast agent, serving as a reference image and illustrating vessel lumen clarity, soft tissue contrast differentiation, and coronary arterial detail obtainable through conventional contrast-enhanced imaging. A third photograph (right) depicts an enhanced coronary imaging representation 412 generated by the system of the present disclosure from the non-contrast CAC scan input without administration of a contrast agent to a patient, illustrating the inference of latent anatomical and structural information including vessel lumen clarity and soft tissue contrast characteristics comparable to those depicted in the traditional CCTA reference image.

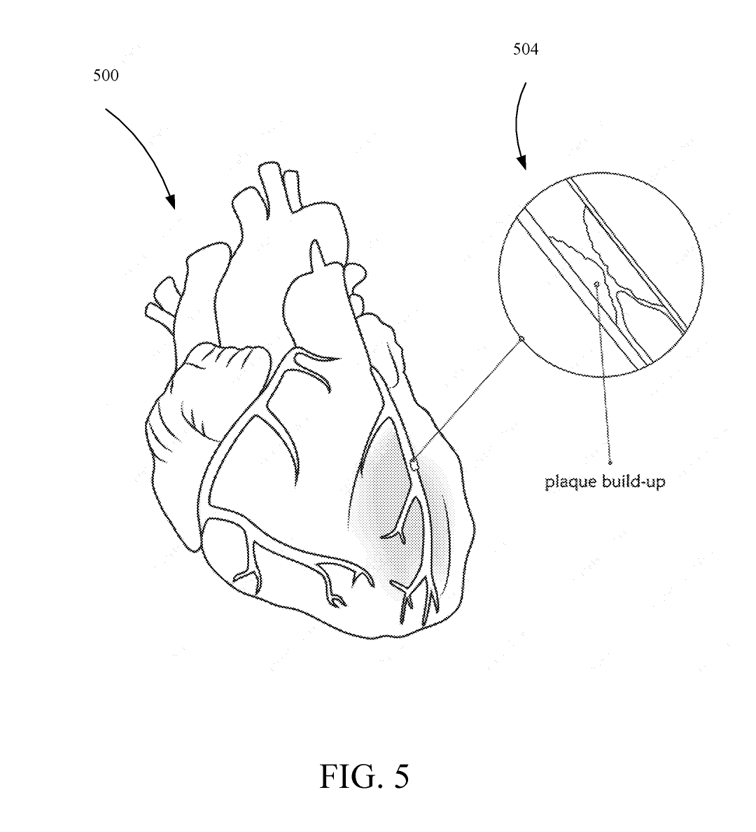

Referring now to FIG. 5, an illustration of an embodiment of a structural assessment output is presented. One or more structures of anatomical region 500 may be identified, including but not limited to: coronary arterial vessel walls; vessel lumens; calcified plaque deposits; non-calcified plaque deposits; mixed plaque formations; fibroatheroma; fibrocalcific plaque; pathological intimal thickening; adaptive intimal thickening; plaque build-up regions; stenotic segments exhibiting luminal narrowing; coronary ostia; branch points within the coronary tree; pericardial boundaries; aortic root structures; or any other anatomically or diagnostically relevant structures within the coronary vasculature. In some embodiments, the structural assessment output may visually delineate one or more of the identified structures with overlaid annotations, color-coded regions, or quantitative measurements to facilitate clinical interpretation.

The structural assessment may include generating classifications of one or more segments 504. Segments 504 may correspond to discrete portions of the coronary arterial tree, including without limitation: left main coronary artery segments; left anterior descending artery segments, including proximal, mid, and distal portions thereof; diagonal branch segments; left circumflex artery segments, including proximal, mid, and distal portions thereof; obtuse marginal branch segments; right coronary artery segments, including proximal, mid, and distal portions thereof; posterior descending artery segments; posterolateral branch segments; ramus intermedius segments; or any other coronary artery segments defined in accordance with a standardized coronary segmentation model. In some embodiments, segments 504 may be identified by applying one or more machine learning models configured to detect coronary ostia, trace coronary centerlines, and delineate segment boundaries based on anatomical landmarks including branch points, vessel diameter transitions, and curvature changes within the coronary tree. In some embodiments, segments 504 may be identified using rule-based anatomical criteria, learned segmentation boundaries, or a combination thereof. Each of segments 504 may be independently assessed for the presence, type, and severity of coronary artery disease findings.

In some embodiments, classifications of one or more segments 504 may include one or more of: plaque type classification, wherein detected plaque within a given segment is classified as calcified plaque, non-calcified plaque, mixed plaque, fibroatheroma, fibrocalcific plaque, pathological intimal thickening, adaptive intimal thickening, or any combination thereof; stenosis severity estimation, wherein luminal narrowing within a given segment is quantified as a percent diameter stenosis, an area stenosis, or both, and assigned a severity classification ranging from no significant stenosis to critical stenosis; vessel geometry metrics, including vessel diameter, wall thickness, lumen area, remodeling index, and centerline geometry along the coronary tree; confidence or uncertainty scores associated with each classification; and high-risk segment flags identifying segments exhibiting one or more indicators of elevated clinical risk, including without limitation critical stenosis, vulnerable plaque morphology, or positive remodeling. In some embodiments, classifications of one or more segments 504 may be generated on a per-vessel-segment basis in accordance with a standardized coronary segmentation model, including but not limited to the Society of Cardiovascular Computed Tomography (SCCT) 18-segment model or any other clinically accepted coronary segmentation scheme. In some embodiments, the structural assessment output may present classifications of one or more segments 504 as overlaid annotations, tabular summaries, color-coded segment maps, or interactive visualizations to facilitate clinical interpretation and reporting.

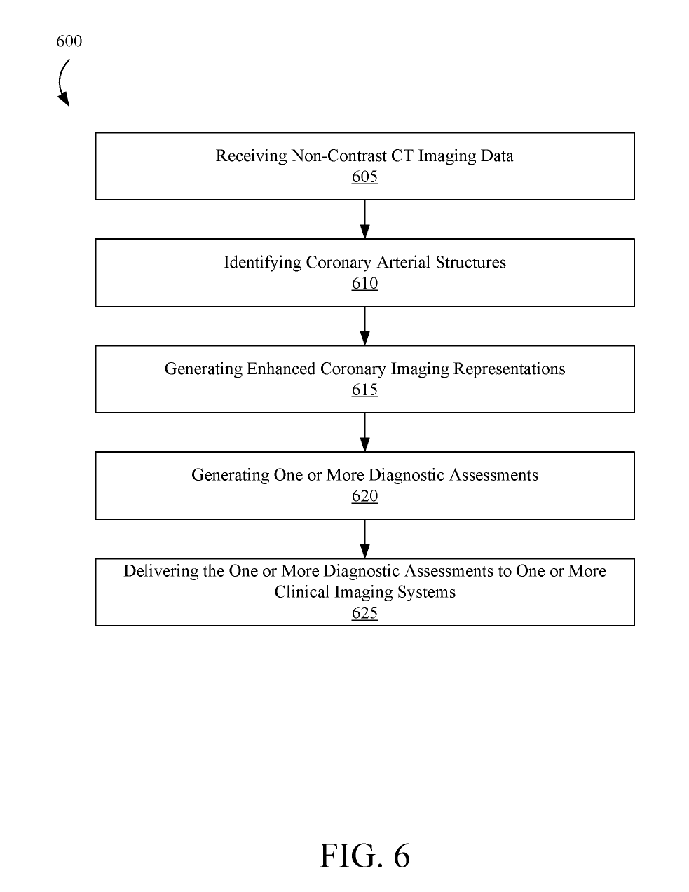

Referring now to FIG. 6, a flowchart of an embodiment of a method 600 of generating enhanced coronary imaging representations from non-contrast CT data is presented. Method 600 may be performed by system 100 of FIG. 1, or by any other suitable computing system comprising one or more processors and one or more non-transitory computer-readable media storing instructions that, when executed by the one or more processors, cause the system to perform the operations described herein. At step 605, method 600 includes receiving non-contrast CT imaging data. The non-contrast CT imaging data may include one or more of coronary artery calcium (CAC) scoring scan data, low-dose cardiac CT data, non-cardiac chest CT data in which coronary structures appear incidentally, multi-energy CT data, or any other CT imaging data acquired without administration of a contrast agent to a patient. The non-contrast CT imaging data may be received in DICOM format or any other suitable medical imaging data format from a CT imaging system, a picture archiving and communication system (PACS), a radiology information system (RIS), or any other clinical imaging data source. In some embodiments, the non-contrast CT imaging data may be preprocessed upon receipt, including one or more of Hounsfield unit windowing and normalization, voxel resampling to a standardized isotropic resolution, field-of-view cropping, motion artifact reduction, noise reduction, and image quality assessment.

At step 610, method 600 includes identifying coronary arterial structures within the non-contrast CT imaging data. In some embodiments, identifying coronary arterial structures may include applying one or more machine learning models configured to detect coronary ostia, trace coronary centerlines, segment the coronary lumen and vessel wall, and identify calcified deposits within the coronary tree. Machine learning models employed at step 610 may include, without limitation, convolutional neural networks, attention-based architectures, graph neural networks, hybrid architectures, or any other architectures configured to perform three-dimensional semantic segmentation of medical imaging data. In some embodiments, identifying coronary arterial structures may further include performing voxel-level segmentation, region-of-interest extraction, centerline extraction, three-dimensional volumetric reconstruction, multi-planar reformatting, or any combination thereof. In some embodiments, a multi-stage pipeline may be employed in which a first model localizes a region of interest within the non-contrast CT imaging data and a second model performs fine-grained segmentation of coronary arterial structures within the localized region.

At step 615, method 600 includes generating enhanced coronary imaging representations from the identified coronary arterial structures and the non-contrast CT imaging data. In some embodiments, generating enhanced coronary imaging representations may include applying one or more trained machine learning models, such as machine learning model 120 of FIG. 1, to infer latent anatomical and structural information from patterns, spatial relationships, and structural cues present within the identified coronary arterial structures and the non-contrast CT imaging data. The enhanced coronary imaging representations may include anatomical or structural information not directly observable in or recoverable through conventional processing of the non-contrast input data, including one or more of: vessel lumen clarity; soft tissue contrast characteristics approximating those obtainable through contrast-enhanced imaging; plaque-related features including identification and characterization of calcified plaque, non-calcified plaque, mixed plaque, fibroatheroma, fibrocalcific plaque, pathological intimal thickening, or adaptive intimal thickening; stenosis severity on a per-vessel-segment basis; perfusion-related vessel characteristics; vessel wall morphology; or any other anatomical, structural, or diagnostic features that are absent from, latent within, or not directly discernible in non-contrast CT acquisitions. In some embodiments, the enhanced coronary imaging representations generated at step 615 may provide clinical information comparable to contrast-enhanced coronary CT angiography (CCTA) without requiring administration of a contrast agent to a patient.

At step 620, method 600 includes generating one or more diagnostic assessments from the enhanced coronary imaging representations. In some embodiments, generating one or more diagnostic assessments may include performing quantitative analysis of coronary artery structures identified within the enhanced coronary imaging representations. The one or more diagnostic assessments may include one or more of: (i) plaque characterization, including classification of detected plaque as calcified, non-calcified, mixed, or other subtypes on a per-vessel-segment basis; (ii) stenosis severity estimation, including quantitative estimation of luminal narrowing expressed as percent diameter stenosis, area stenosis, or both, with associated severity classification; (iii) vessel geometry metrics, including vessel diameter, wall thickness, lumen area, remodeling index, and centerline geometry along the coronary tree; (iv) coronary tree visualization, including interactive two-dimensional and three-dimensional visualizations with overlaid structural findings; (v) uncertainty quantification, including confidence scores or uncertainty estimates associated with the enhanced coronary imaging representations and the diagnostic assessments; and (vi) longitudinal comparison, including co-registration and comparative analysis across multiple time-point studies to identify changes in plaque burden, stenosis severity, and vessel geometry. In some embodiments, the one or more diagnostic assessments may include additional diagnostic outputs, biomarkers, or predictive indicators derived from the enhanced coronary imaging representations.

At step 625, method 600 includes delivering the one or more diagnostic assessments to one or more clinical imaging systems. In some embodiments, delivering the one or more diagnostic assessments may include transmitting the enhanced coronary imaging representations and associated diagnostic outputs to one or more of a picture archiving and communication system (PACS), a radiology information system (RIS), an electronic health record system, a radiology reporting platform, or any combination thereof, via DICOM-compatible interfaces or any other suitable communication protocol. Output formats may include, without limitation: DICOM-formatted imaging series; structured reports compatible with Health Level Seven Fast Healthcare Interoperability Resources (HL7 FHIR) standards; web-based interactive viewer outputs; and PDF-formatted diagnostic summary reports. In some embodiments, delivering the one or more diagnostic assessments may further include providing a programmatic application programming interface (API) configured to return processed outputs to third-party platforms, including downstream AI-based analysis tools. In some embodiments, method 600 may be performed within clinically relevant timeframes without requiring contrast agents, intravenous access, specialist staffing, post-procedure monitoring, or modification to existing CT hardware. In some embodiments, one or more steps of method 600 may be performed in a different order than presented, may be performed in parallel, or may be omitted, without departing from the scope of the present disclosure.

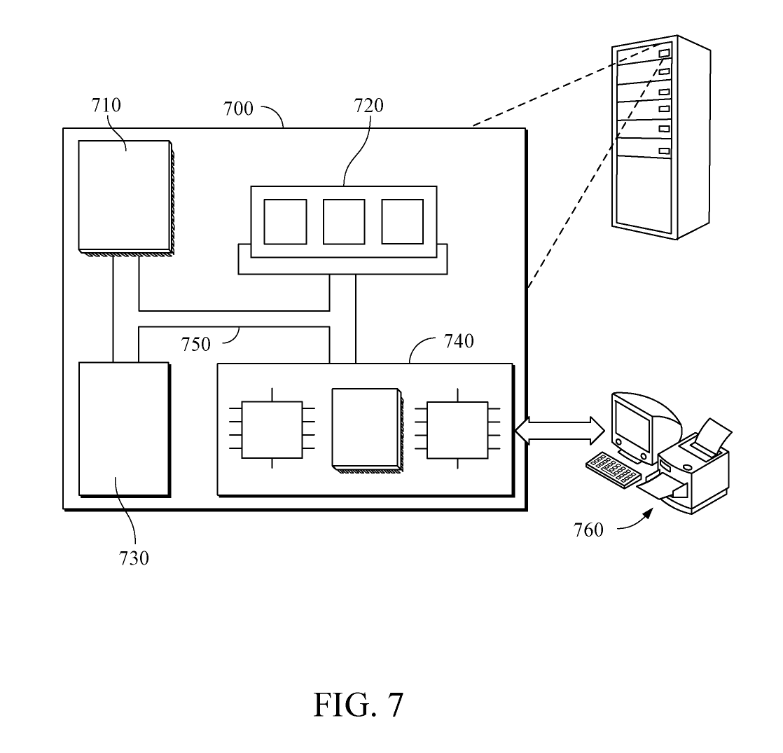

Referring now to FIG. 7 is a block diagram of an example computer system 700 that may be used in implementing the technology described in this document. General-purpose computers, network appliances, mobile devices, or other electronic systems may also include at least portions of the system 700. The system 700 includes a processor 710, a memory 720, a storage device 730, and an input/output device 740. The apparatus may include disk storage and/or internal memory, each of which may be communicatively connected to each other. The system 700 may include a processor 710. The processor 710 may enable both generic operating system (OS) functionality and/or application operations. In some embodiments, the processor 710 and the memory 720 may be communicatively connected. As used in this disclosure, “communicatively connected” means connected by way of a connection, attachment, or linkage between two or more elements which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device.

Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. In some embodiments, the processor 710 may include any computing device as described in this disclosure, including without limitation a microcontroller, microprocessor, digital signal processor (DSP) and/or system on a chip (SoC) as described in this disclosure. The processor 710 may include, be included in, and/or communicate with a mobile device such as a mobile telephone or smartphone. The processor 710 may include a single computing device operating independently, or may include two or more computing device operating in concert, in parallel, sequentially or the like. Two or more computing devices may be included together in a single computing device or in two or more computing devices. The processor 710 may interface or communicate with one or more additional devices as described below in further detail via a network interface device. Network interface device may be utilized for connecting the processor 710 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

The processor 710 may include but is not limited to, for example, a computing device or cluster of computing devices in a first location and a second computing device or cluster of computing devices in a second location. The processor 710 may include one or more computing devices dedicated to data storage, security, distribution of traffic for load balancing, and the like. The processor 710 may distribute one or more computing tasks as described below across a plurality of computing devices, which may operate in parallel, in series, redundantly, or in any other manner used for distribution of tasks or memory between computing devices. The processor 710 may be implemented using a “shared nothing” architecture in which data is cached at the worker, in an embodiment, this may enable scalability of system 700 and/or processor 710.

With continued reference to FIG. 7, processor 710 and/or a computing device may be designed and/or configured by memory 720 to perform any method, method step, or sequence of method steps in any embodiment described in this disclosure, in any order and with any degree of repetition. For instance, the processor 710 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The processor 710 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Each of the components 710, 720, 730, and 740 may be interconnected, for example, using a system bus 750. The processor 710 is capable of processing instructions for execution within the system 700. In some implementations, the processor 710 is a single-threaded processor. In some implementations, the processor 710 is a multi-threaded processor. In some implementations, the processor 710 is a programmable (or reprogrammable) general purpose microprocessor or microcontroller. The processor 710 is capable of processing instructions stored in the memory 720 or on the storage device 730.

The memory 720 stores information within the system 700. In some implementations, the memory 720 is a non-transitory computer-readable medium. In some implementations, the memory 720 is a volatile memory unit. In some implementations, the memory 720 is a non-volatile memory unit.

The storage device 730 is capable of providing mass storage for the system 700. In some implementations, the storage device 730 is a non-transitory computer-readable medium. In various different implementations, the storage device 730 may include, for example, a hard disk device, an optical disk device, a solid-date drive, a flash drive, or some other large capacity storage device. For example, the storage device may store long-term data (e.g., database data, file system data, etc.). The input/output device 740 provides input/output operations for the system 700. In some implementations, the input/output device 740 may include one or more network interface devices, e.g., an Ethernet card, a serial communication device, e.g., an RS-232 port, and/or a wireless interface device, e.g., an 802.11 card, a 3G wireless modem, or a 4G/5G wireless modem. In some implementations, the input/output device may include driver devices configured to receive input data and send output data to other input/output devices, e.g., keyboard, printer and display devices 760. In some examples, mobile computing devices, mobile communication devices, and other devices may be used.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one

example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

What is claimed is:

1. A computer-implemented system configured to generate enhanced anatomical representations from non-contrast medical imaging data, the system comprising:
one or more processors; and
one or more non-transitory computer-readable media storing instructions that, when executed by the one or more processors, cause the system to:
receive non-contrast medical imaging data;
identify anatomical structures or regions of interest within the imaging data; and
generate enhanced representations of the identified anatomical structures by inferring or reconstructing anatomical features not directly observable in or recoverable through conventional processing of the non-contrast input data;
wherein the enhanced representations include information not directly observable in the non-contrast input.

2. A computer-implemented system configured to derive enhanced coronary representations from non-contrast computed tomography (CT) data, wherein the enhanced representations include anatomical or structural information not directly observable in the input imaging data, the system comprising:
one or more processors; and
one or more non-transitory computer-readable media storing instructions that, when executed by the one or more processors, cause the system to:
receive non-contrast CT imaging data;
identify coronary arterial structures within the imaging data;
generate enhanced coronary representations by inferring anatomical or structural information not directly observable in the non-contrast input; and
deliver the enhanced coronary representations to one or more clinical systems.

3. A system for generating contrast-equivalent, enhanced, augmented, or diagnostically enriched coronary imaging representations from non-contrast CT data, the system comprising:
one or more preprocessing modules configured to receive non-contrast CT imaging data and produce normalized imaging data;
one or more coronary artery localization and segmentation modules configured to identify coronary arterial structures within the normalized imaging data;
one or more image enhancement and synthesis modules configured to generate enhanced coronary imaging representations from the non-contrast input and the identified coronary arterial structures, wherein the enhanced representations include information inferred from but not directly observable in the non-contrast input data;
one or more structural assessment modules configured to generate diagnostic outputs from the enhanced coronary imaging representations; and
one or more output interface modules configured to deliver the enhanced representations and diagnostic outputs to one or more clinical imaging systems;
wherein the system generates enhanced coronary imaging representations without requiring administration of a contrast agent to a patient.

4. A method for generating enhanced coronary imaging representations from non-contrast CT data, the method comprising:
receiving non-contrast CT imaging data from a CT imaging system;
preprocessing the imaging data to produce normalized imaging data;
identifying coronary arterial structures within the normalized imaging data using one or more machine learning models;
generating enhanced coronary imaging representations from the non-contrast input using one or more trained transformation models, wherein the enhanced representations include anatomical or structural information inferred from but not directly observable in the non-contrast input;
generating one or more diagnostic assessments from the enhanced coronary imaging representations; and
delivering the enhanced representations and diagnostic assessments to one or more clinical imaging systems;
wherein the method generates enhanced coronary representations without requiring administration of a contrast agent to a patient.

5. A non-transitory computer-readable medium storing instructions that, when executed by one or more processors, cause the processors to:
receive non-contrast CT imaging data in DICOM format;
normalize and preprocess the imaging data;
apply one or more machine learning models to identify coronary arterial structures;
apply one or more trained transformation models to generate enhanced coronary imaging representations by inferring information not directly observable in the non-contrast input;
generate structural diagnostic outputs; and
transmit the enhanced representations and outputs to one or more clinical systems via DICOM-compatible interfaces.

6. The system of claim 3, wherein the one or more image enhancement and synthesis modules are trained on datasets comprising paired and/or unpaired imaging data, including but not limited to ex-vivo cadaver CT data, in-vivo clinical CAC scan data, in-vivo CCTA data, or any combination thereof.

7. The system of claim 3, wherein the one or more image enhancement and synthesis modules perform transformation using at least one of: direct image-to-image transformation, latent space reconstruction, feature-level enhancement, hybrid multi-stage transformation, or any other method of deriving enhanced representations from non-contrast imaging data.

8. The system of claim 3, wherein the one or more structural assessment modules generate one or more of: plaque type classification per vessel segment, stenosis severity estimation per vessel segment, vessel geometry metrics, coronary tree visualization, uncertainty scores, longitudinal comparative analysis, or additional diagnostic outputs, biomarkers, or predictive indicators.

9. The system of claim 8, wherein plaque type classification includes one or more of: calcified plaque, non-calcified plaque, mixed plaque, fibroatheroma, fibrocalcific plaque, pathological intimal thickening, and adaptive intimal thickening.

10. The system of claim 3, wherein the one or more output interface modules integrate with at least one of: a PACS system, a RIS system, an electronic health record system, a radiology reporting platform, or a programmatic API.

11. The system of claim 3, wherein the system is deployable in at least one of: a cloud-based configuration, an on-premise configuration, an edge computing configuration, or a combination thereof.

12. The system of claim 3, wherein the preprocessing module is configured to accept at least one of: cardiac CT data, low-dose cardiac CT data, non-cardiac chest CT data in which coronary structures appear incidentally, and multi-energy CT data.

13. The system of claim 3, wherein the system generates enhanced coronary representations and structural assessment outputs within clinically relevant timeframes, without new CT scanner hardware or contrast agent administration.

14. The system of claim 3, wherein the system is configured for use in at least one of: population-level cardiac screening, pre-imaging triage, assessment of patients contraindicated for contrast-enhanced imaging, emergency cardiac assessment, incidental coronary finding assessment, and longitudinal disease monitoring.

15. The method of claim 4, wherein generating enhanced coronary imaging representations comprises applying transformation models trained on datasets comprising paired and/or unpaired imaging data, including but not limited to ex-vivo cadaver CT imaging data with co-registered ground truth and in-vivo clinical CAC and CCTA scan pairs.

16. The method of claim 4, wherein generating one or more diagnostic assessments comprises generating at least one of: plaque type classification, stenosis severity estimation, vessel geometry metrics, coronary tree visualization, confidence scores, longitudinal comparative analysis, or additional diagnostic outputs, biomarkers, or predictive indicators derived from non-contrast imaging data.

17. The system of claim 2, wherein the system is further configured to generate additional diagnostic outputs, biomarkers, or predictive indicators derived from non-contrast imaging data, including outputs beyond those expressly identified herein.

18. The system of claim 3, wherein the enhanced coronary imaging representations provide clinically useful diagnostic information sufficient for coronary artery disease assessment, without requiring administration of a contrast agent.
